(19) United States
(12) Patent Application Publication
(10) Pub. No.: US 2023/0189796 A1
(43) Pub. Date: Jun. 22, 2023

(54) COMPOSITE MATERIAL

(71) Applicant: Mendel University in Brno, Brno (CZ)

(72) Inventors: Vojtech ADAM, Brno (CZ); Zuzana BYTESNIKOVÁ, Brno (CZ); Ales EICHMEIER, Brno (CZ); Robert POKLUDA, Brno (CZ); Jakub PECENKA, Brno (CZ); Lukas RICHTERA, Brno (CZ); Dorota TEKIELSKA, Brno (CZ)

(73) Assignee: Mendel University in Brno, Brno (CZ)

(21) Appl. No.: 17/926,902

(22) PCT Filed: May 18, 2021
(86) PCT No.: PCT/EP2021/063087

§ 371 Date: Nov. 21, 2022

(30) Foreign Application Priority Data
May 20, 2020 (CZ) PV 2020-287

(51) Int. Cl. A01N 25/08; A01N 59/16; B82Y 5/00; B82Y 40/00

(52) U.S. Cl. CPC A01N 25/08; A01N 59/16; B82Y 5/00; B82Y 40/00; A01N 2300/00

(57) ABSTRACT

The present invention relates to composite materials based on a graphene-like substrate, decorated with nanoparticles of at least two different metals. The composite materials show superior antibacterial activity in a variety of contexts, notably in phytosanitary applications.

The present invention relates to composite materials, methods for the preparation of such a material, and a number of uses of the material.

### BACKGROUND

Metal nanoparticles have gained attention due to their unique physical, chemical and biological properties. Silver nanoparticles in particular possess a broad spectrum of antibacterial, antifungal and antiviral properties. Silver nanoparticles have the ability to penetrate bacterial cell walls, changing the structure of cell membranes, sometimes resulting in cell death. Their efficacy is due not only to their nanoscale size but also to their large surface area to volume ratio. They increase the permeability of cell membranes, produce reactive oxygen species, and interrupt replication of deoxyribonucleic acid by releasing silver ions (Int J Nanomedicine. 2020; 15: 2555-2562).

Silver nanoparticles (Ag NPs) are known for their excellent antibacterial properties against a wide range of microorganisms, and are used in various fields including food packaging, water treatment, dressing materials, and antibacterial agents. Their disadvantage, which reduces their effectiveness, is their aggregation and oxidation. Copper-containing biocides are attractive in agriculture for their antibacterial effects and low cost. Copper nanoparticles (Cu NPs) have better antibacterial properties than copper salts due to their large surface area and resistance to washing out during watering or rain. However, if Cu NPs are agglomerated, their effect decreases rapidly. Graphene oxide (GO) is a unique material with excellent chemical and physical properties that has been used in many industries, environmental applications, biomedical equipment, and agriculture due to its biocompatibility and low cost. In agriculture, GO has found use, for example, in the slow release of fertilizers and bactericides, or adsorption of heavy metals and toxins.

### DESCRIPTION OF THE PRIOR ART

Karanikolos et al. (ACS Appl. Mater. Interfaces 2016, 8, 41, 27498-27510) disclose silver and copper monometallic and bimetallic nanoparticles which were grown in situ on the surface of graphene, in turn produced by chemical vapor deposition using ferrocene as precursor and further functionalized to introduce oxygen-containing surface groups. The antibacterial performance of the resulting hybrids was evaluated against Escherichia coli cells. It was found that both Ag- and Cu-based monometallic graphene composites significantly suppress bacterial growth, yet the Ag-based ones exhibit higher activity compared to that of their Cu-based counterparts.

Karanikolos et al. (Langmuir 2018, 34, 11156-11166) discloses ion-based, metal/graphene hybrid structures comprising surface-bound silver and copper mono-ionic and bi-ionic species on functionalized graphene, without involvement of nanoparticles. It was found that the bi-ionic Ag/Cu-graphene materials exhibited superior performance compared to that of the mono-ionic analogues owing to the synergistic action of the combination of the two different metal ions on the surface and the enhancing role of the graphene support, whereas all ion-based systems performed superiorly compared to their NP-based counterparts of the same metal type and concentration.

The present invention addresses these and other problems of the prior art.

### SUMMARY OF THE INVENTION

According to a first embodiment, the invention provides a composite material having a graphene-based substrate, with nanoparticles of at least two different metals bonded thereto, wherein the loading of metal ions is at least 10% based on the weight of the material.

According to a second embodiment, the invention provides a process for the preparation of a composite material, comprising the steps of:

i. providing a mixture of graphene oxide and a solution containing salts of at least two different metals;

ii. adding a reducing agent to the mixture of step i.

According to a third embodiment, the invention provides a pesticidal composition comprising a composite material of the invention together with suitable excipients and/or adjuvants.

According to a fourth embodiment, the invention provides a composite material of the invention for use as a medicine.

According to a fifth embodiment, the invention provides the use of a composite material of the invention as a microbicide.

According to a sixth embodiment, the invention provides a material comprising a microbicidal amount of a composite material of the invention.

### BRIEF DESCRIPTION OF THE DRAWINGS

FIG. 1: Schematic representation of the rGO-Cu—Ag nanocomposite synthesis procedure from GO and SEM images of the starting material GO (A), the prepared rGO-Cu—Ag nanocomposite (B) and EDS analysis of rGO-Cu—Ag showing the elemental composition (C).

FIG. 2: rGO-Cu—Ag composite synthesis apparatus.

FIG. 3: In vitro assays demonstrating the synergistic effect of rGO-Cu—Ag according to Example 4.

FIG. 4: Comparison of the effectiveness of the rGO-Cu—Ag composite with Kocide 2000 (active ingredient copper hydroxide 53.8%=35% by weight of metallic copper) on the spotting of tomatoes. The frequency of symptoms of black speck on tomatoes 7th and 14th day after inoculation, the column was a statistically significant difference (value of P≤0.05, Duncan's test) are among them distinguished letters.

FIG. 5: Untreated and uninoculated control (31 days after sowing)

FIG. 6: Negative control—nanocomposite treated plants (31 days after sowing)

FIG. 7: Plants treated with nanocomposite and subsequently inoculated with Xanthomonas euvesicatoria (31 days after sowing)

FIG. 8: Positive control—plants inoculated with Xanthomonas euvesicatoria (31 days after sowing)

FIG. 9: Results of relative expression of selected genes in Example 7: btub=reference control of the reference gene for betatubulin, PR1=pathogen esis—related protein encoding the gene for glucan endo-1,3-beta-D-glucosidase, pop=sequence encoding the precursor for polyphenol oxidase, cat=sequence encoding the catalase gene, prq=sequence encoding the gene for the formation of the basic form of b-1,3-glucanase III. class, tomq=sequence encoding the gene for the acid form of b-1,3-glucanase III. class.

### DEFINITIONS

As used herein, the term “nanoparticulate”, “nanoparticle”, and the like refer to particles between 0.1 and 500 nanometres (nm) in diameter, preferably between 1 and 100 nm. When applied to collections of particles, the term applies to those ensembles having at least 80% (such as at least 90%, at least 99% or at least 99.5%) of particles having the stated diameter.

As used herein, the term “diameter”, when applied to a non-spherical particle or collection of such particles, refers to the diameter of the sphere that has the same volume or mass as that particle.

As used herein, the term “graphene-based” refers to a material which is an allotrope of carbon consisting of a single layer of atoms arranged in a two-dimensional honeycomb lattice, or a derivative of such a material. The skilled person will appreciate that the term includes graphene itself.

### DESCRIPTION OF THE PREFERRED EMBODIMENTS

### Substrate

The substrate used for the composite materials of the invention is a graphene-based material. In some embodiments, the substrate may be graphene itself. Alternatively, the substrate may be a derivative of graphene, such as hydrogenated graphene (graphane), fluorinated graphene (fluorographene), oxidized graphene (graphene oxide), and graphene introduced with acetylenic chains (graphyne and graphdiyne). A particularly preferred graphene-based material is graphene oxide, or reduced graphene oxide.

As will be well known to the skilled person, graphene oxide is a compound of carbon, oxygen, and hydrogen in variable ratios, and the structure and properties of graphene oxide depend on particular synthesis method and degree of oxidation. Prior art methods (Karanikolos et al., vide supra) have commenced with graphene produced by chemical vapour deposition (CVD), and oxidized the graphene thus obtained with a mixture of concentrated nitric and sulphuric acids. However, this results in a material capable only of binding low amounts of metal.

Surprisingly, the inventors have found that a far superior loading of metal nanoparticles, such as an order of magnitude better, can be obtained through use of graphene prepared via an alternative route. The preferred substrate for use in the nanoparticles of the invention is graphene oxide prepared by oxidation of graphite.

It is known that different methods of oxidation of graphite produce graphene oxide having significantly different properties, in particular in terms of the carbon to oxygen atomic ratio. A number of oxidants may be used for the oxidation of graphite in the preparation of the substrate. For example, Brodie's method (treatment of a slurry of graphite with fuming nitric acid with potassium chlorate for 4 days at 60° C.), the Staudenmeier-Hoffman-Hamdi method (treatment of a slurry of graphite with mixture of sulfuric acid and nitric acid in the presence of potassium chlorate at high temperature, at about 90° C. for 4 days), Hummer's method (addition of potassium permanganate to a solution of graphite, sodium nitrate, and sulfuric acid), Tour's method (use of a high concentration of KMnO₄ and excess of H₂SO₄ and H₃PO₃), and various modifications thereof.

A preferred graphene oxide substrate is prepared by oxidation of graphite with KMnO₄ in concentrated sulphuric acid, followed by termination of the reaction by addition of hydrogen peroxide. This material has been found to be particularly advantageous for use in subsequent steps, and in particular is capable of achieving a high loading of metal ions.

In preferred embodiments, the graphene-based material is a reduced graphene oxide. A reduced graphene oxide is a graphene oxide as defined above, which has undergone a reduction reaction.

### Metal Nanoparticles

Nanoparticles of at least two different metals are bonded to the substrate, according to the present invention. As used herein, the term “bonded” encompasses covalent and non-covalent (e.g. ionic) boding.

“Metal” should be understood by the skilled person, however, for the avoidance of doubt, the term encompasses alkali metals (lithium, sodium, potassium, rubidium, caesium, and francium), alkaline earth metals (beryllium, magnesium, calcium, strontium, barium, and radium), transition metals (scandium, titanium, vanadium, chromium, manganese, iron, cobalt, nickel, copper, yttrium, zirconium, niobium, molybdenum, technetium, ruthenium, rhodium, palladium, silver, hafnium, tantalum, tungsten, rhenium, osmium, iridium, platinum, gold, rutherfordium, dubnium, seaborgium, bohrium, and hassium), post-transition metals (aluminium, zinc, gallium, cadmium, indium, tin, mercury, thallium, lead, bismuth, polonium, and astatine), lanthanides (lanthanum, cerium, praseodymium, neodymium, promethium, samarium, europium, gadolinium, terbium, dysprosium, holmium, erbium, thulium, ytterbium, and lutetium) and actinides (actinium, thorium, protactinium, uranium, neptunium, plutonium, americium, curium, berkelium, californium, einsteinium, fermium, mendelevium, nobelium, and lawrencium).

It is preferred that the at least two different metals are selected from the same group of the periodic table, more preferably from group 1 to 15. Still more preferably, the at least two different metals are selected from Group 11 of the periodic table. Still more preferably, the metals are selected from copper (Cu), silver (Ag) and gold (Au).

The term “at least two” encompasses 2, 3, 4, 5 and higher integers. Preferably, the composite material of the invention comprises two metals. Most preferably, the two metals are copper and silver.

In the case where nanoparticles of two different metals are present, the atomic ratio of these may be between 9:1 and 1:9. Preferably, the ratio is between 5:1 and 1:5, more preferably between 2:1 and 1:2, such as between 3:2 and 2:3. In the case where the two metals are copper and silver, the atomic ratio of copper to silver may be between 9:1 and 1:9. Preferably, the ratio of copper to silver is between 5:1 and 1:5, more preferably between 2:1 and 1:2, such as between 3:2 and 2:3, including about 1:1.

A preferred aspect of the invention is that the composite materials are substantially free of iron. In this context, “substantially free” means that the particles contain less than 5%, such as less than 1%, preferably less than 0.1% w/w of Fe. Iron has deleterious effects on the biological properties of the composite materials.

In some embodiments, the rGO-Cu—Ag (reduced graphene oxide having nanoparticles of copper and silver) materials of the invention is characterised by the infrared spectrum. In particular, materials of the invention show the presence of bands at 1105 cm⁻¹, 1339 cm⁻¹, 2998 cm⁻¹ and 3549 cm⁻¹.

### Processes for Preparation

In preferred embodiments, the at least two metals are deposited simultaneously onto the graphene-based substrate. This is in contradistinction to the methods of the prior art (e.g. Karanikolos et al., vide supra) which teach the necessity of sequentially depositing metals.

In processes according to the invention, the graphene-based substrate (or a precursor thereof) is placed in suspension or solution together with salts (preferably soluble salts) of the at least two metal ions. The solvent or suspending medium is preferably aqueous, although non-aqueous liquids and mixtures of water with non-aqueous liquids is also contemplated.

The graphene-based substrate may be present in its final form, i.e., it is not chemically changed during the deposition reaction. Alternatively, and preferably, it may be provided as a precursor. In the cases where the graphene-based substrate is reduced graphene oxide, the precursor is graphene oxide prepared by any of the methods described above.

The salts of the at least two metal ions may comprise the metals in a higher oxidation state than is present in the final nanoparticulate material. For example, in the case of copper, the salt employed during the reaction may be a copper (II) salt or copper (I), which is reduced to copper (0). In the case of silver, the salt employed during the reaction may be a silver (I) salt, which is reduced during the reaction to silver (0).

Careful selection of the salts of the at least two metal ions is necessary, as certain mixtures will be incompatible and cause a precipitation reaction. For example, silver (I) nitrate is incompatible with halide salts, as silver halide would precipitate from the solution.

Having provided the mixture of the graphene-based substrate (or a precursor thereof) and the salts of the at least two metal ions, to the said mixture is added a reducing agent. Many suitable reducing agents will be available, including boranes, hydrogen, metal hydrides, etc. Preferred reducing agents are metal borohydrides, such as sodium borohydride, and metal aluminohydrides, such as lithium aluminium hydride. Sodium borohydride is preferred.

In a preferred embodiment, the precursor of the graphene-based substrate and at least one of the at least two metal ions are reduced concomitantly. In a preferred embodiment, wherein the precursor of the graphene-based substrate is graphene oxide, reduced graphene oxide (rGO) is formed on reduction, at the same time as at least one of the at least two metal ions is reduced. In a particularly preferred embodiment, copper (II) is reduced to copper (0) and silver (I) is reduced to silver (0) at the same time as graphene oxide is reduced to rGO.

The present process is advantageous compared to known processes for preparing graphene-based materials comprising nanoparticles of more than one metal, because they can be introduced in a single step. Furthermore, the organisation of the ions on the surface of the substrate is very different from materials wherein the ions are introduced stepwise, in a way that is not completely understood, but leads to significant advantages in terms of the loading (on a w/w basis) of metal ions that can be achieved, and the properties of the resultant materials.

### Biological Activity

The composite materials of the present invention have a range of biological activities. In particular, the composite materials display antibacterial effects which are useful in both a medical and phytosanitary context.

The composite materials display antimicrobial effects, and in particular antibacterial effects against a range of clinically significant pathogens. Pathogens which may be controlled by the materials of the present invention include gram-negative bacteria and gram positive bacteria, such as Achromobacter xylosoxidans, Acinetobacter baumannii, Actinomyces, Actinomyces israelii, Aeromonas species, Bacillus species, Bacteroides fragilis, Bacteroides melaninogenicus, Bartonella species, Bordetella pertussis, Borrelia species, Brucella species, Burkholderia species, Campylobacter, Capnocytophaga species, Chlamydophila pneumoniae, Chlamydophila psittaci, Citrobacter species, Clostridium species, Corynebacterium species, Coxiella burnetii, Ehrlichia species, Eikenella corrodens, Enterobacter species, Enterococcus faecalis, Enterococcus faecium, Escherichia coli, Francisella tularensis, Fusobacterium necrophorum, Gardnerella vaginalis, Haemophilus species, Helicobacter Pylori, Klebsiella species, Lactobacillus species, Legionella species, Leptospira species, Listeria monocytogenes, Moraxella catarrhalis, Morganella species, Mycobacteria species, Mycoplasma species, Neisseria species, Nocardia species, Pasteurella multocida, Peptostreptococcus species, Porphyromonas gingivalis, Propionibacterium acnes, Proteus species, Providencia species, Pseudomonas aeruginosa, Salmonella species, Serratia marcescens, Shigella species, Staph epidermidis, Staph hominis, Staph. haemolyticus, Staphylococcus aureus, Staphylococcus saprophyticus, Stenotrophomonas maltophilia, Streptococcus agalactiae, Streptococcus anginosus group, Streptococcus pneumoniae, Streptococcus pyogenes (Groups A, B, C, G, F), Treponema pallidum, and Vibrio species.

Composite materials may be formulated for treatment of bacterial infections of humans or animals suitable for any desired route of administration, in particular via enteral, parenteral, or topical routes.

Suitable dosages of the composite materials may be determined. As a guide, for an oral formulation, a dosage range of 0.1-100 mg/kg is envisaged. r

Oral formulations of the invention also comprise a bulk diluent or filler. Diluents act as fillers in pharmaceutical tablets to increase weight and improve content uniformity. Natural diluents include starches, hydrolyzed starches, and partially pregelatinized starches. Common diluents include anhydrous lactose, lactose monohydrate, and sugar alcohols (polyols) such as sorbitol, xylitol and mannitol.

The oral formulations of the invention may comprise various other pharmaceutical adjuvants and excipients. Pharmaceutically acceptable excipients are well known to those skilled in the art and are disclosed for example, in Staniforth, U.S. Pat. No. 6,936,277, and Lee, U.S. Pat. No. 6,936,628, each of which is incorporated herein by reference in its entirety for all purposes. Excipients such as diluents, binders, glidants, and lubricants are added as processing aids to make the tableting operation more effective. Still other types of excipients enhance or retard the rate of disintegration of the tablet, improve the taste of the tablet (for example, sweetening agents), or impart a colour or flavour to the tablets.

One or more lubricants may be added to a tablet formulation comprising the particulate product of the present invention to prevent the formulation from sticking to the punches during tablet manufacture. Suitable lubricants include, for example, fatty acids, fatty acid salts, and fatty acid esters such as magnesium stearate, calcium stearate, stearic acid, sodium stearyl fumarate, hydrogenated vegetable oil and the like. Lubricants may typically comprise about 0.1 wt % to about 3.0 wt % or about 0.5 wt % to about 2 wt %, most preferably about 1.4% to about 1.6%, such as about 1.5% of the formulation. Magnesium stearate is preferred.

Antiadherents may be utilized to prevent sticking of the tablet formulation to the punch face and die wall. They are used in combination with magnesium stearate when sticking is a problem. Commonly used antiadherents are corn-starch and talc.

One or more binders in addition to the particulate product of the present invention may be added to further modify the cohesive qualities of the powdered material(s). Suitable additional binders include starch, microcrystalline cellulose, and sugars such as sucrose, glucose, dextrose, and lactose. Partially pregelatinized maize starch is preferred.

The amount of binder is preferably from 1 to 20%, more preferably from 5 to 15%, more preferably from 8 to 12% such as about 10% w/w.

Additionally, one or more disintegrants may also be included in the tablet formulation to ensure that the tablet has an acceptable dissolution rate in an environment of use (such as the gastrointestinal tract). The disintegrant breaks up the tablets and the granules into particles of active and excipients. Super-disintegrants such as croscarmellose sodium, sodium starch glycolate, or crospovidone may also be employed. Of these, sodium starch glycolate is preferred.

The amount of disintegrant is preferably from 1 to 10%, more preferably from 2 to 8%, more preferably from 3 to 5% such as about 4% w/w.

One or more glidants may be used in the tablet formulation to improve flow. Because of the shape and size of the particles, glidants improve flow in low concentrations. They may be mixed in the final tablet formulation in dry form. Suitable glidants include, for example, alkali metal stearates, colloidal silicon dioxide (including materials sold under the brand names CAB-O-SIL®, SYLOID®, and AEROSIL®), and talc.

Topical formulations are also contemplated. In such formulations, the composite materials are formulated as a cream, gel, foam, lotion or ointment in a suitable vehicle for direct application to the skin or wound of an afflicted human or animal.

### Pesticidal Activity

The composite materials of the invention show notable activity against a range of plant pathogens, in particular bacteria of the genera Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma. The composite materials show remarkable activity against Xanthomonas species, in particular Xanthomonas euvesicatoria. As such, the composite materials find use in the treatment and/or prevention of bacterial diseases in a range of crops, such as citrus fruits (post-harvest and foliar treatments), stone fruits (cherry, peach and plum), berries and small fruit (blackberry, blueberry, cranberry, grapes, raspberry and strawberry), tropical fruits with inedible peel (banana, mango and papaya), bulb vegetables (bulb onion, spring onion and leeks), brassica vegetables (broccoli, Brussels sprouts, cabbage, cauliflower and kohlrabi), fruiting vegetables (cucumber, gherkin, melon, summer squash, pepper and tomato), lettuce, legume vegetables (beans and peas), pulses (soybeans), root and tuber vegetables (beetroot, carrot, chicory, potato, radish and sugar beet), stalk and stem vegetables (artichokes, asparagus, celery, witlof and chicory), cereal grains (barley, oat, rye, triticale, wheat, maize and rice), tree nuts (almonds, pecans and pistachios), oil seeds (cottonseed, peanuts and sunflower), herbs (basil, chives, parsley and mint), peanut hay, soya bean forage and hay, straw, fodder and forages of cereal grains (barley, oat, rye, triticale, wheat, maize and rice), sugar beet tops, dried herbs (basil, chives, parsley and hops), and almond hulls.

The composite materials are particularly useful in the treatment and/or prevention of bacterial diseases of plants of the genus Solanum, including tomatoes, potatoes and aubergines. In a preferred embodiment, the composite materials are useful in treatment and/or prevention of Xanthomonas euvesicatoria infestation of tomatoes.

It is notable that the pesticidal compositions of the invention do not stress crop plants, and exhibit desirably low levels of phytotoxicity.

The agricultural compositions according to the invention comprises, as active material, at least one composite material as a mixture with solid or liquid vehicles which are acceptable in agriculture and/or surface-active agents which are also acceptable in agriculture. In particular, the inert and conventional vehicles and the conventional surface-active agents can be used. These compositions cover not only compositions which are ready to be applied to the crop to be treated by means of a suitable device, such as a spray device, but also commercial concentrated compositions which have to be diluted before application to the crop. The composite material is denoted as active material.

These compositions can also contain any kind of other ingredients such as, for example, protective colloids, adhesives, thickening agents, thixotropic agents, penetrating agents, stabilizing agents, sequestering agents and the like. More generally, the composite materials can be combined with all the solid or liquid additives corresponding to the conventional formulating techniques.

Generally, the compositions according to the invention usually contain from 0.05 to 95% (by weight) of active material, one or more solid or liquid vehicles and, optionally, one or more surface-active agents.

In the present account, the term “vehicle” denotes a natural or synthetic, organic or inorganic material with which the active material is combined to facilitate its application on the aerial parts of the plant. This vehicle is thus generally inert and it must be acceptable in agriculture, especially on the treated plant. The vehicle can be solid (clays, natural or synthetic silicates, silica, resins, waxes, solid fertilizers and the like) or liquid (water, alcohols, especially butanol, and the like).

The surface-active agent can be an emulsifying, dispersing or wetting agent of ionic or nonionic type or a mixture of such surface-active agents. There may be mentioned, for example, salts of polyacrylic acids, salts of lignosulphonic acids, salts of phenolsulphonic or naphthalenesulphonic acids, polycondensates of ethylene oxide with fatty alcohols or with fatty acids or with fatty amines, substituted phenols, (especially alkylphenols or arylphenols), salts of esters of sulphosuccinic acids, taurine derivatives (especially alkyltaurates), phosphoric esters of polyoxyethylenated phenols or alcohols, esters of fatty acids and of polyols, and the derivatives of the above compounds containing sulphate, sulphonate and phosphate functional groups. The presence of at least one surface-active agent is generally indispensable when the active material and/or the inert vehicle is/are not soluble in water and the carrier agent for application is water.

The compositions for agricultural use according to the invention can thus contain the active material within very wide limits, ranging from 0.05% to 95% (by weight). Their surface-active agent content is advantageously of between 5% and 40% by weight. Unless otherwise specified percentages in this specification, including the accompanying claims, are by weight.

These compositions according to the invention are themselves in fairly diverse, solid or liquid forms.

There may be mentioned, as forms of solid compositions, the powders for dusting (with an active material content which can range up to 100%) and the granules, especially those obtained by extrusion, by compacting, by impregnation of a granulated support, or by granulation from a powder (the active material content in these granules being between 0.5 and 80% for the latter cases), the tablets or effervescent tablets.

The agricultural composition according to the invention can also be used in the form of powders for dusting; it is also possible to use a composition comprising 50 g of active material and 950 g of talc; it is also possible to use a composition comprising 20 g of active material, 10 g of finely divided silica and 970 g of talc; these constituents are mixed and milled and the mixture is applied by dusting.

There may be mentioned, as forms of liquid compositions or those intended to constitute liquid compositions at the time of application, solutions, in particular water-soluble concentrates, emulsions, suspension concentrates, aerosols, wettable powders (or powder to be sprayed), pastes or gels.

The suspension concentrates, which can be applied by spraying, are prepared so as to obtain a stable fluid product which does not settle out and they generally contain from 10 to 75% of active material, from 0.5 to 15% of surface-active agents, from 0.1 to 10% of thixotropic agents and from 0 to 10% of suitable additives, such as antifoaming agents, corrosion inhibitors, stabilizing agents, penetrating agents and adhesives and, as vehicle, water or an organic liquid in which the active material is insoluble or nearly insoluble: certain organic solid materials or inorganic salts can be dissolved in the vehicle to aid in preventing sedimentation or as antigels for water.

Various wettable powder (or powder to be sprayed) compositions are given here as examples:

### Wettable Powder 1

composite material of Example 2 50%
ethoxylated fatty alcohol (wetting agent) 2.5%
ethoxylated phenylethylphenol (dispersing agent) 5%
chalk (inert vehicle) 42.5%

### Wettable Powder 2

composite material of Example 2 10%
C13 branched-type synthetic oxo alcohol, ethoxylated with 8 to 10 molecules of ethylene oxide (wetting agent) 0.75%
neutral calcium lignosulphonate (dispersing agent) 12%
calcium carbonate (inert filler) qs for 100%

### Antimicrobial Materials

In one aspect, the invention pertains to a material comprising a microbicidal amount of a composite material of the invention. A number of materials can incorporate composite materials of the invention and hence attain long-lasting antibacterial properties.

Amongst the materials that can be thus treated, fabrics (such as cotton, wool and synthetic fibres), plastics (such as polyethylene, polypropylene, polyamides, polyesters, PET, polypropylene, polystyrene, polyurethanes, polyvinyl chloride, and acrylonitrile butadiene styrene), paper, construction board (low-, medium and high-density fibreboard), concrete, cement and metals.

The composite material of the invention can be incorporated into the base material (e.g. plastic) by incorporation of an appropriate amount at its time of manufacture. Suitably, the treated material comprises from 0.1 to 10% by weight of the composite material of the invention.

The materials thus produced find particular application in situations where bacterial growth is a problem, including, but not limited to, wound dressings, medical devices, surgical utensils, and food preparation equipment.

Alternatively, the composite material of the invention may be used as a surface treatment. As such, the composite material may be incorporated into a paint, varnish or other applicable formulation and applied to the surface of an item by spraying, dipping or painting.

In yet another aspect, the composite material of the invention may be incorporated into a sanitizing product, such as a cleaning spray. Typically, from 0.01 to 2% by weight of the composite material of the invention is incorporated in such a product, with other ingredients typical in such applications, such as surfactants, fragrances and colours.

### Particular Aspects of the Invention

This section relates to particular aspects of the invention.

In a particular aspect, the present invention relates to a process for the preparation of reduced graphene oxide in a nanocomposite with silver nitrate and copper acetate. This composite can be used to reduce the expression of genes specific for various plant pathogens. The nanocomposite material may be prepared by reacting copper acetate and silver nitrate with a graphene oxide solution and then reducing them with sodium borohydride to form nanoparticles.

The application of the rGO-Cu—Ag nanocomposite to plants does not affect the expression of any tested gene, the plants do not show stress, but for selected proteins the application of this nanocomposite leads to a reduction of gene expression in plants treated with nanoparticles and then infected with bacteria. The nanocomposite does not stress plants, on the contrary, it reduces the expression of genes specific for various plant pathogens.

In a particular, the present invention relates to a process for the preparation of a nanocomposite material based on reduced graphene oxide, silver nitrate and copper acetate, which comprises the following steps:

i) preparing graphene oxide by reacting graphite with sulfuric acid and potassium permanganate; the reaction product is then mixed with an aqueous solution of hydrogen peroxide and washed to form a suspension of graphene oxide in water;

ii) preparing a suspension of graphene oxide from step i) in an aqueous solution of silver nitrate and copper acetate, the molar ratio of silver nitrate to copper acetate being 1:1, and wherein the weight ratio of graphene oxide to silver nitrate to copper acetate is 1:8, 5:9.1;

iii) the suspension from step ii) is reduced with a reducing agent to form a nanocomposite material (rGO-Cu—Ag) containing reduced graphene oxide, in the structure of which silver and copper nanoparticles are incorporated.

By nanoparticles is meant particles with a size ranging from 1 to 100 nm.

The advantage of the graphene oxide in step i) is prepared so that the graphite flakes (preferably, 5 g) added to concentrated H₂SO₄ (preferably 670 ml, 96%) and then added KMnO₄ (preferably 30 g). The reaction mixture thus prepared is stirred vigorously for at least 10 days. The oxidation of the graphite is terminated by the addition of an aqueous solution of H₂O₂ (preferably 250 ml, 30% by weight) with vigorous stirring and cooling. The graphite oxide formed is washed with 1 M HCl and then with water to a constant pH (3-4).

Preferably, the slurry graphene oxide, silver nitrate and copper acetate in step ii) is prepared by mixing aqueous solutions of silver nitrate and copper acetate at a final concentration of 5 mM AgNO₃ and 5 mM Cu(OAc)₂ and then adding this solution dropwise under vigorous stirring to an aqueous suspension of graphene oxide from step i) at a concentration of 5 g/l.

In a preferred embodiment, the reducing agent is sodium borohydride. Preferably, the reducing agent in step iii) is used in amount of at least eight times the weight of the graphene oxide in suspension.

The present invention further relates to a nanocomposite material based on reduced graphene oxide, silver nitrate and copper acetate, obtainable by the process of the present invention. This material contains reduced graphene oxide (rGO), in the structure of which copper and silver nanoparticles are incorporated.

The present invention further relates to a composition for the protection of plants, in particular tomatoes, but also peppers and other plants, against Xanthomonas euvesicatoria. The preparation contains an aqueous suspension of a nanocomposite material based on reduced graphene oxide, silver nitrate and copper acetate (rGO-Cu—Ag), preparable according to the process of the present invention. The concentration preferably is RGO-Cu—Ag of at least 0.01 g/mL, preferably at least 0.05 g/mL, even more preferably at least 0.1 g/mL, preferably 5 g/mL. The upper limit of the concentration of rGO-Cu—Ag in the preparation is not limited, the preparation may be in the form of a concentrate, used for later dilution by the user. The preparation may further contain excipients. Excipients are solvents (especially water), surfactants (surfactants—nonionic, cationic or anionic surfactants), emulsifiers, dispersants, humectants, wetting agents, stabilizers (e.g. vegetable oils or epoxidized vegetable oils), defoamers (e.g. silicone oil), preservatives, viscosity agents, binders, adhesives, and optionally fertilizers.

The composition of the present invention can be applied to whole plants, leaves, plant organs or plant cells in tissue cultures.

The present invention also relates to the use of a nanocomposite material based on reduced graphene oxide, silver nitrate and copper acetate in agriculture. The advantage of this material can be used for treating plants against Xanthomonas euvesicatoria, particularly for protecting tomatoes and peppers. The treatment can be performed by spraying a suspension of nanocomposite on the leaves and stems of plants at their early stage of development.

It is advisable to treat preventively growth, especially in the localities where the bacteria has already been recorded. The first treatment should take place in the phase of 4 true leaves, at a concentration of 500 μg/l (50 l/m² or 500 l/ha). Further treatment should then be performed according to the presence of the pathogen.

### EXAMPLES

The invention is described in more detail below by means of exemplary embodiments, which, however, in no way limit other possible embodiments within the scope of the claims.

### Materials

Chemicals used in this study, unless otherwise stated, were purchased from Sigma-Aldrich (St. Louis, Mo., USA). The deionized water was prepared using reverse osmosis equipment Aqual 25 (Aqua Osmotic, Tisnov, Czech Republic). The deionized water was further purified by using the apparatus MilliQ Direct QUV, equipped with the UV lamp (Aqua Osmotic, Tisnov, Czech Republic). The resistance was 18.2 MΩ. The pH was measured using pH meter WTW inoLab (Weilheim, Germany).

### Example 1: Preparation of Graphene Oxide (GO)

5 g of graphite flakes (Sigma-Aldrich and 100 mesh, ≥75% min) were added to concentrated H₂SO₄ (670 ml) followed by 30 g of KMnO₄. The reaction mixture thus prepared was stirred vigorously. After 10 days, oxidation of graphite was terminated by the addition of a solution of H₂O₂ (250 ml, 30% by weight of O₂), Penta, Chrudim, Czech Republic). The H₂O₂ solution was added dropwise slowly and the whole mixture was cooled vigorously and stirred. The graphite oxide formed was washed with 61 of 1 M HCl and then with Milli-Q water (total volume used 10 l) until a constant pH value was reached (3-4).

### Example 2: Preparation of rGO-Cu—Ag Composite

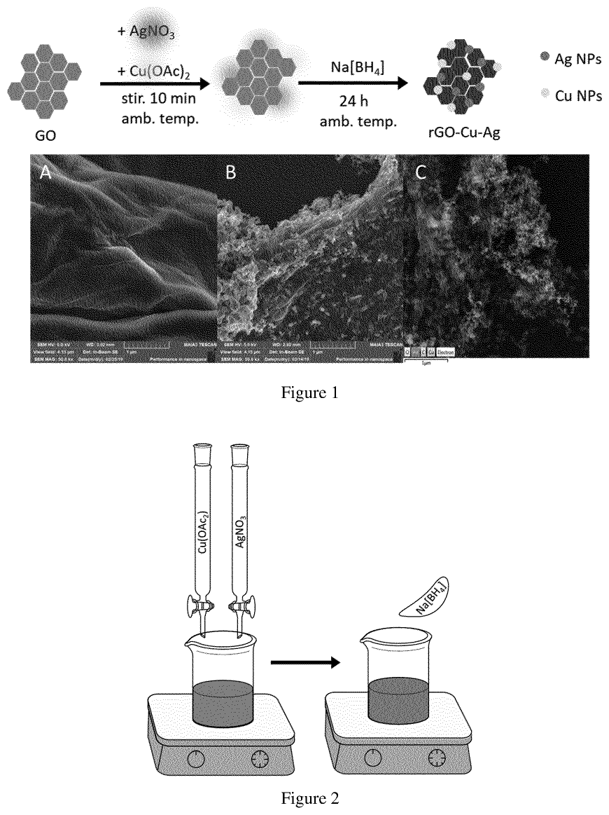

Aqueous solutions of silver nitrate (25 mL, 10 mM) and copper acetate (25 mL, 10 mM) were added dropwise to aqueous GO from example 1 (1 mL, 5 g/L) with vigorous stirring. Then, the reducing agent Na[BH₄] (40 mg) was slowly added to the reaction mixture, and the resulting mixture was stirred vigorously for 24 hours at room temperature. The prepared nanocomposite was washed three times with Milli-Q water. The final volume was adjusted to 10 ml. The composite concentration was determined to be 5 g/l. The prepared composite was characterized by SEM (scanning electron microscopy) and EDS (energy dispersive X-ray spectroscopy)—see FIG. 1.

The dispersed samples were diluted 1:20 with Milli-Q with water and then allowed to dry on a silicone plate at room temperature (20-25° C.). The plate was then analysed using a MAIA 3 SEM (TESCAN Ltd, Brno, Czech Republic), In-Beam SE detector, 5 keV, distance 3 mm.

The elemental composition of the composite according to the present invention was studied using the energy-dispersive X-ray spectroscopy (EDS) method, EDX detector on MIRA 2 SEM (TESCAN Ltd, Brno, Czech Republic), In-Beam SE 15 keV, distance 15 mm.

Fourier-transform infrared spectroscopy (FTIR) analysis of GO indicated bands corresponding to vibrations C—O (1050 cm⁻¹), C═C (1631 cm⁻¹), C═O (1737 cm⁻¹), C—H (2925 cm⁻¹). The 3322 cm⁻¹ wide band corresponded to O—H vibrations. Analysis showed RGO-Cu—Ag contained bands, corresponding to C—O (1105 cm⁻¹ and 1339 cm⁻¹) and O—H (2998 cm⁻¹ and 3549 cm⁻¹). After the reduction by Na[BH₄], O—H groups are retained and additional alcohols are introduced as a result of hydrolysis of boron esters.

### Example 3: Preparation of rGO-Ag and rGO-Cu Composites to Confirm the Synergistic Effect of Ag and Cu Ions

An aqueous solution of silver nitrate (50 mL, 10 mM) for the synthesis of rGO-Ag, or copper acetate (50 mL, 10 mM) for the synthesis of rGO-Cu was added to an aqueous solution of GO (1 mL, 5 g/L) with vigorous stirring. Subsequently, the reducing agent Na[BH₄] (40 mg) was added slowly to the reaction mixture, and the resulting mixture was stirred vigorously for 24 hours at room temperature. The prepared nanocomposite was washed three times with Milli-Q water. The final volume was adjusted to 10 mL.

### Example 4: Antibacterial Activity of rGO-Cu—Ag Composite Vs. rGO-Ag and rGO-Cu

The antibacterial properties of the rGO-Cu—Ag composite against the bacterial strain X. euvesicatoria were investigated, and compared with the antibacterial properties of rGO-Ag and rGO-Cu.

Antibacterial activity was assessed by determining the number of living bacteria according to colony-forming units (CFU). Bacterial strain X. euvesicatoria no. 2968 was provided by a collection of microorganisms at the National Collection of Plant Pathogenic Bacteria (NCPPB, London, UK) and were grown in Luria-Bertani (LB) medium (Sigma-Aldrich) at 28° C. overnight. Subsequently, the bacteria were adjusted to an optical density of 600 nm (OD₆₀₀) and subsequently serially diluted with LB medium. The resulting suspensions were mixed in a 1:1 ratio with the suspensions of rGO-Cu—Ag, rGO-Cu and rGO-Ag prepared in Examples 2 and 3, to final concentrations of 5, 0.1 and 0.01 μg/mL. The suspensions were incubated for 24 hours at 28° C. and shaken at 110 rpm. Sterile distilled water was used instead of the nanocomposite suspension to prepare the untreated control.

To determine the number of living bacteria the “pour plate method” (a method for calculating forming bacteria present in the liquid sample) was used. 100 μL samples from each mixture were diluted in ten-fold series. 100 mL of each diluted sample was pipetted into the centre of a sterile Petri dish (diameter 90 mm) and a sample was added to a sterile plate count agar (Himedia, Mumbai, India) (44-46° C.) which was mixed with the sample. The samples were then cooled to room temperature and incubated at 28° C. for 40 hours. Bacterial colonies formed were then counted and expressed as a percentage of CFU from the untreated control.

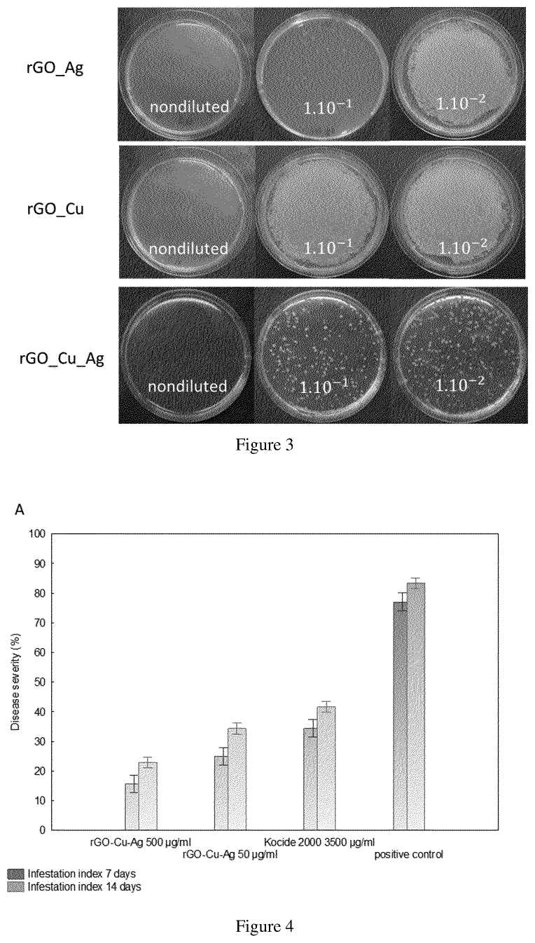

As shown in Table 1 and FIG. 3, the bacterial growth was inhibited by all three composites investigated during use concentration nanocomposites 5 ppm/mL. At lower concentrations, differences in the antibacterial properties of the composites were observed, with the rGO-Cu—Ag composite of the present invention showing the greatest antibacterial effects.

TABLE 1
Antibacterial effects of composites on the growth of X. euvesicatoria
strain bacteria. Results are expressed as a percentage of growing
bacteria in comparison with untreated control.

| Composite | | | |
|---|---|---|---|
| concentration | Bacterial growth (%) | | |
| (μg/mL) | rGO-Ag | rGO-Cu | rGO-Cu—Ag |
| 5 | 0 | 0 | 0 |
| 0.1 | 16.28 | 93.8 | 30.12 |
| 0.01 | 96.28 | 97.57 | 33.57 |

From Table 1 and FIG. 3 it is evident that RGO-Cu and RGO-Ag exhibited antibacterial activity only at a concentration of 5 μg/mL (RGO-Cu) and 0.1 μg/mL (rGO-Ag), the rGO-Cu—Ag composite of the present invention showed a synergistic effect and effectively suppressed bacterial growth even at a concentration of 0.01 μg/mL.

The results of antibacterial in vitro tests were the basis for subsequent tests on plants.

### Example 5: Greenhouse Experiment of the Effect of rGO-Cu—Ag Composite on Tomato Plants

For the experiment, the “Mandat” cultivar of tomato was used. Plants were grown in 280 ml flasks containing standard substrate TS 4 (Klasmann-Deilmann GmbH Geeste, Germany) and maintained between 22 and 26° C. at ≥70% relative humidity. For the experiment plants at the stage of four leaves were used, on which was spray applied a nanocomposite according to the present invention, prepared in Example 2 and diluted to concentrations of 50 and 500 ppm. After 24 hours, the plants were sprayed with a bacterial suspension of X. euvesicatoria (1×10⁸ CFU). After inoculation, the plants were covered with polythene bags for a period of 48 hours in order to increase humidity. For the positive control the nanocomposite was replaced with a sterile aqueous salt solution. The negative control was treated only with nanocomposite at a concentration of 500 μg/mL and sterile aqueous saline was used instead of the bacteria solution. 0.35% by weight was chosen as another treatment method. A standard commercial preparation Kocide® 2000 (DuPont, Wilmington, Del., USA), which contains as active ingredient copper hydroxide 53.8 wt. %=35 wt. % metallic copper. Ten plants were subjected to each of the treatments described above, and the whole experiment was repeated twice. Symptom analysis was performed on days 7 and 14 after inoculation. Bacterial symptoms were evaluated on a four-point scale:

0—healthy leaves without symptoms

1—low proportion of symptoms (1 to 3 spots on the leaf)

2—one third of the leaf surface infected

3—high incidence of symptoms (more than a third of the leaf surface infected).

On the basis of the evaluation of symptoms was determined by the degree of severity of the infection, the percentage (DS disease severity) using the following formula:

$$DS ( \% ) = \frac{\begin{matrix}\sum ( number of plants in a disease scale point \times \\ disease scale point )\end{matrix}}{( total no . of plants \times maximum disease scale point )} \times 100$$

### Example 6: Treatment of Plants with rGO-Cu—Ag Composite

Four variants were used to ascertain the effect of nanocomposite application:
untreated control,
plants treated with the nanocomposite prepared in Example 2,
plants treated with the nanocomposite prepared in Example 2 and subsequently inoculated with Xanthomonas euvesicatoria,
plants not treated with nanocomposite and inoculated with Xanthomonas euvesicatoria

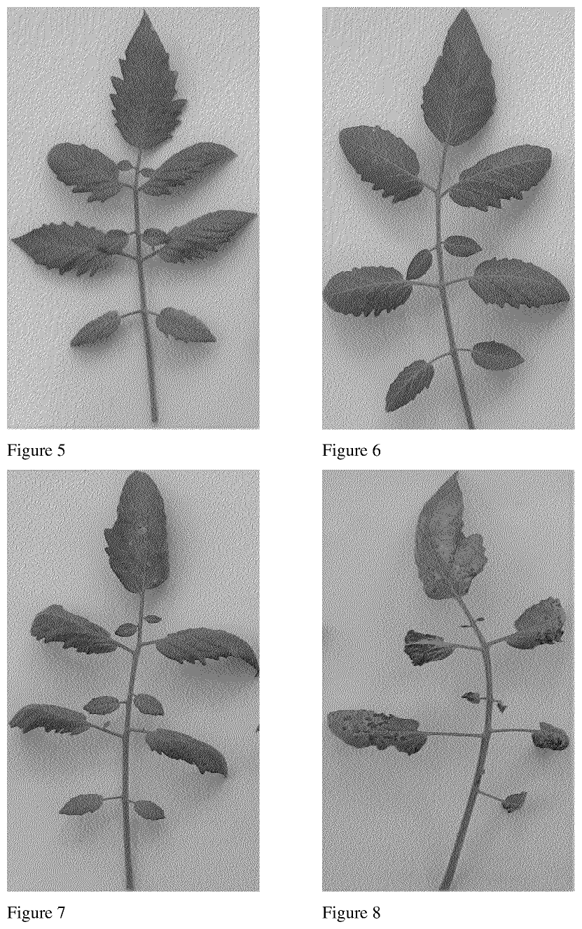

The nanocomposite material prepared according to Example 2 with a volume of 10 ml and a concentration of 5 g/L was diluted with water to a composite concentration of 500 μg/L and then sprayed at a concentration (50 ml/m²) on each tomato plant in phase 4 true leaves. The bacterial strain Xanthomonas euvesicatoria in a suspension of 1.108 CFU (colony forming units) was used for inoculation. Plants were analysed 31 days after sowing: untreated and uninoculated control (FIG. 5), negative control—nanocomposite-treated plants (FIG. 6), nanocomposite-treated plants subsequently inoculated with Xanthomonas euvesicatoria (FIG. 7) and positive control—plants inoculated bacteria Xanthomonas euvesicatoria (FIG. 8). It can be seen from the figures that the rGO-Cu—Ag nanocomposite effectively inhibits the development of the disease caused by the Xanthomonas euvesicatoria strain and, in addition, no phytotoxic effect of the nanocomposite on the studied plants was observed.

### Example 7: Effect of rGO-Cu—Ag on Relative Gene Expression in Tomato Plants

To monitor the effect of rGO-Cu—Ag nanocomposite application, 4 plant variants were used, as in Example 6 (variant 1=untreated control, variant 2=nanocomposite treated plants prepared in Example 2, variant 3=nanocomposite treated plants prepared in Example 2 and subsequently inoculated with Xanthomonas euvesicatoria, variant 4=plants not treated with nanocomposite and inoculated with Xanthomonas euvesicatoria).

Total RNA was isolated to monitor the effect of rGO-Cu—Ag. Tomato plants of the ‘Mandat’ variety were used to extract total RNA, and RNA was extracted using the Spectrum Plant Total RNA Kit (Sigma-Aldrich, USA). RNA extraction was performed immediately after the first occurrence of black spot symptoms from 6-week-old plant tissue, with sampling taking place simultaneously from all 4 variants (for variants, see FIG. 9). The symptomatic parts of the plants, namely the leaves and parts of the stems, were removed. The plant tissue was homogenized using liquid nitrogen, 100 mg of homogenized plant tissue each being used to extract RNA from one sample. To test the expression of the gene protocols for a total of 11 genes were optimized, of which two genes for reference, then six genes were selected on the grounds of their expression in a result of different types of stress in a grid if n.

The expression of the control gene b tub (betatubulin) was the same in all four variants 1-4, relative quantity (RQ) of 1.5. Quantification of PR1 gene expression (pathogenesis-related protein 1 encoding the glucan endo-1,3-beta-D-glucosidase gene) gave RQ values of var. 1 (0), var. 2 (0), var. 3 (3,2) and var. 4 (13.8) (see FIG. 9). Nanocomposite treatment reduced PR1 gene expression 4.3-fold. Very similar expression results have been reported for the other pop genes (sequence encoding the polyphenol oxidase precursor), cat (sequence encoding the catalase gene) and prq (sequence encoding the basic form of class III b-1,3-glucanase) associated with defensive reaction of plants. The reduction in the expression of genes associated with plant biotic stress confirms the antagonistic effect of rGO-Cu—Ag.

Further aspects of the invention are disclosed and defined in the appended claims.

1. A composite material having a graphene-based substrate, with nanoparticles of at least two different metals bonded thereto, wherein the loading of metal ions is at least 10% based on the weight of the material.

2. A composite material according to claim 1 wherein the first and second metals are selected from copper, silver and gold.

3. A composite material according to claim 1 wherein the first metal is silver, and the second metal is copper.

4. A composite material according to claim 1, wherein the amount of iron present in the material is less than 5% w/w, such as less than 1%.

5. A composite material according to claim 1, wherein the graphene-based material is a single-layer material.

6. A composite material according to claim 1, wherein the substrate is reduced graphene oxide (rGO).

7. A process for the preparation of a nanoparticulate material, comprising the steps of:
i. providing a mixture of graphene oxide and a solution containing salts of at least two different metals;
ii. adding a reducing agent to the mixture of step i.

8. A process according to claim 7 wherein the at least two metals are copper and silver.

9. A process according to claim 8 wherein the copper salt is a copper (II) salt, preferably copper (II) acetate.

10. A process according to claim 7, wherein the silver salt is silver (I) nitrate.

11. A process according to claim 8, wherein the atomic ratio of silver to copper is between 9:1 and 1:9, such as between 2:1 and 1:2, preferably about 1:1.

12. A process according to claim 7, wherein the reducing agent is a borohydride, preferably sodium borohydride.

13. A process according to claim 7, wherein the graphene oxide is obtained by oxidation of graphite, preferably with sulfuric acid and potassium permanganate.

14. A process according to claim 7 comprising further steps of washing and/or purification.

15. A composite material obtainable by a process according to claim 7.

16. A pesticidal composition comprising a composite material according to claim 1 together with suitable excipients and/or adjuvants.

17. A pesticidal composition according to claim 16 further comprising one or more fungicides, herbicides, insecticides, acaricides or fertilizers.

18.-22. (canceled)
